(19) United States
(12) Patent Application Publication
(10) Pub. No.: US 2021/0248515 A1
(43) Pub. Date: Aug. 12, 2021

(54) ASSISTED LEARNING WITH MODULE PRIVACY

(71) Applicant: Regents of the University of Minnesota, Minneapolis, MN (US)

(72) Inventors: Jie Ding, Plymouth, MN (US); Xun Xian, Minneapolis, MN (US); Xinran Wang, Plymouth, MN (US)

(21) Appl. No.: 17/248,845

(22) Filed: Feb. 10, 2021

Related U.S. Application Data
(60) Provisional application No. 62/975,348, filed on Feb. 12, 2020.

(51) Int. Cl. G06N 20/00; G06N 5/04

(52) U.S. Cl. CPC G06N 20/00; G06N 5/04

(57) ABSTRACT

Techniques are disclosed for assisted learning with module privacy. In one example, a module creates a learner unit by fitting, into a first fitted label set, an initial label set using a first learning technique, a first machine learning model, and a first feature set, send, to at least one module that provides assisted learning, first statistical information defined by at least one residual from fitting the first fitted label set, wherein each module is operative to fit, into a second fitted label set, the first statistical information using at least one second learning technique, a second machine learning model, and a second feature set, receives second statistical information from the at least one module, the second statistical information being defined by at least one residual from fitting the second fitted label set, and updates the learner unit by fitting, into a third fitted label set, the second statistical information.

### ASSISTED LEARNING WITH MODULE PRIVACY

This application claims the benefit of U.S. Provisional Application No. 62/975,348, filed Feb. 2, 2020, the entire content of which is incorporated by reference herein.

### TECHNICAL FIELD

This disclosure generally relates to machine learning architectures.

### BACKGROUND

Machine learning can often be defined as a data analysis technology for knowledge to be extracted by a machine, without any explicit definition to conduct the same, based on a series of observations. In general, machine learning refers to a number of scientific principles (e.g., pattern recognition principles) that determine if the machine is capable of learning from a data corpus and of reproducing repeatable actions with higher reliability and efficient decision making. In the era of big data with exploding size and complexity, machine learning technologies have successfully taken advantage of the richness of available data to facilitate industrial development and/or human experience. To illustrate the ubiquity of machine learning, mobile applications frequently make suggestions to users based on previous searches of the user. As one example, a mobile application may suggest a restaurant based upon previous user searches.

A machine learning architecture, in general, refers to an artificial intelligence platform from which a number of machines learn from each other and/or from external sources. The basic idea is to train machines on how to learn and make decisions without explicit inputs from users. In this architecture, one machine may play the role of a user while another machine may play the role of a service such that the user machine receives some intelligence from the service machine. The effectiveness of a conventional machine learning architecture often depends upon the richness of the corpus of training data.

### SUMMARY

In general, the present disclosure describes techniques for assisted learning in a machine learning architecture. As described herein, technologies implementing these techniques may achieve a level of data privacy beyond what is possible in conventional machine learning architectures, without sacrificing quality of any gained intelligence.

Successful conventional machine learning architectures provide intelligence from user data sets but often require disclosure of that data. Concerns of data security and privacy have led to more stringent regulations on the use of data in machine learning. There is considerable interest in designing machine learning architectures that facilitate not only accuracy, but also privacy and data security. In addition, there is also a growing demand for protecting the learner units that manage data.

The techniques for assisted learning in a machine learning architecture, as described herein, may provide one or more technical advantages or improvements that provide at least one practical application. The techniques enable module privacy which, instead of protecting the data alone, protects the privacy on data and model as a black-box. These techniques also improve upon a learning quality of a learner unit. Some techniques utilize a simple linear regression algorithm to train and construct a machine learning model and a learner unit (e.g., a learner unit function).

In the context of a machine learning architecture having a network of remote computing devices operating as modules, the techniques described herein introduce a new level of privacy that protects not only data but also algorithms for each learner unit in a network of learner units. Each learner unit can choose to assist others, or each learner unit receives assistance from others, where the assistance is realized by iterative communications of essential statistics. The communication protocol for assisted learning is designed in a way that protects both types of learner units and benefit the learning performance. The machine learning architecture also leads to a new concept of a machine learning market, which includes learner units and assisting communications (possibly for rewards).

In one example, this disclosure describes a method that includes: creating, by processing circuitry of a computing device, a learner unit by fitting, into a first fitted label set, an initial label set using at least one first learning technique, a machine learning model, and a first feature set; sending, by the processing circuitry of the computing device, to at least one module in a machine learning architecture, first statistical information defined by at least one first residual from fitting the first fitted label set, wherein the at least one module executes on at least one remote computing device, wherein the at least one module is operative to fit, into a second fitted label set, the first statistical information using at least one second learning technique and a second feature set; receiving, by the processing circuitry of the computing device, and from the at least one module, second statistical information that is defined by at least one second residual from fitting the second fitted label set; and updating, by the processing circuitry of the computing device, the learner unit by fitting, into a third fitted label set, the second statistical information using the at least one first learning technique and the machine learning model.

In another example, this disclosure describes a computing device and a non-transitory computer-readable medium comprising instructions to implement any method described herein. In one example, the disclosure describes a computing device for assisted learning with module privacy. In one example, processing circuitry of the computing device creates a learner unit by fitting, into a first fitted label set, an initial label set using at least one first learning technique, a first machine learning model, and a first feature set, sends to at least one module in a machine learning architecture first statistical information defined by at least one residual from fitting the first fitted label set, wherein at least one module runs on at least one remote computing device, wherein at least one module is operative to fit, into a second fitted label set, the first statistical information using at least one second learning technique, a second machine learning model, and a second feature set, receives second statistical information from the at least one module, the second statistical information being defined by at least one residual from fitting the second fitted label set, and updates the learner unit by fitting, into a third fitted label set, the second statistical information using the at least one first supervised learning technique and the first machine learning model.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIGS. 1A-B are block diagrams illustrating example architectures having at least two operations for modules to exchange statistical information and build machine learning models, in accordance with one or more techniques of the disclosure.

FIG. 2 is a block diagram illustrating an example computing device within one or more of the example architectures of FIGS. 1A and/or 1B, in accordance with one or more techniques of the disclosure.

FIG. 3 is a flowchart illustrating an example training process for a learner unit by a computing device of the example architecture of FIGS. 1A or 1B, in accordance with one or more techniques of the disclosure.

FIG. 4 is a block diagram illustrating a relationship between assisted learning and predictive performance as achieved by an example architecture of FIG. 1A or 1B, in accordance with one or more techniques of the disclosure.

FIG. 5A is an illustration of an assisted learning protocol and FIG. 5B is an illustration of a model generate by the assisted learning protocol of FIG. 5A, in accordance with one or more techniques of the disclosure.

Like reference characters refer to like elements throughout the figures and description.

### DETAILED DESCRIPTION

Conventional machine learning architectures provide intelligence from user data but at the cost of disclosing at least some of that data. This disclosure may be purposeful or inadvertent. Typically, a machine learning architecture transmits user data to a data center for further processing. In some cases, an adversary can deduce elements of the user data by requesting certain services related to that data. For at least this reason, conventional architectures achieve technological advancement at the cost of data privacy. This can be a hindrance for both service providers and users (e.g., data analysts), since transmitting user data requires sophisticated encryption against potential attacks and combining data in one basket may be inherently associated with a trustiness issue. Protecting privacy while maximally using available data has been an urgent problem in the era of big data. Concerns of data security and privacy have led to more stringent regulations on the use of data in machine learning. For instance, the European Union's General Data Protection Regulation (GDPR) requires data curators to use more plain language for privacy agreements, and to explain how the algorithms make a particular decision based on users' data. There is considerable interest in designing machine learning architectures that facilitate not only accuracy, but also privacy, data security, and fairness.

State-of-the-art technology ensuring privacy and fairness usually focuses on protecting users' data. However, there is also a growing demand for protecting the learner units who manage data. For example, consider that a health insurance company and a bank collect different features from a large group of people; the bank has information such as deposit, salary and debt, while the health insurance has various medical records. If the health insurance company wants to develop a new insurance product with high return, it is beneficial for the health insurance company to know the financial statues of the targeted clients. Yet, the bank will not directly disclose any individual-level data even if they are perturbed. There exists an incentive to both parties that the bank provides services that do not directly transmit data but still provide relevant information for the insurance company to facilitate machine learning services.

A relevant concern for the bank is the possibility that its developed model is to be reconstructed if an adversary keeps querying. If such a reconstruction occurs, it can be even worse than data release from the bank's perspective, since its core advantage is often the learned black-box model rather than data itself. For example, in financial market, data can be accessed by many algorithmic traders, but the core advantage of a successful trader is a sophisticated algorithm being deployed. In the context of fairness, a user may decide to provide key statistics to assist others' learning while hiding sensitive features and other data.

In the following description, the present disclosure describes technology for a machine learning architecture having a configuration of entity systems operating as modules where each module may be a service module that provides assisting learning to or a user module that receives assisting learning from the other. Regarding assisted learning, the present disclosure may refer to improving a particular module's machine learning performance using information (e.g., statistics) from one or more other modules. As described herein, modules in the machine learning architecture implement a technique to ensure data and algorithm privacy (e.g., module privacy and other privacy concepts), enabling these modules to provide services and/or assisted learning without disclosing any proprietary information (e.g., models). Module privacy, as a concept, refers to protecting the privacy of an entity system's proprietary model in addition to protecting the entity's data and may also be known as model privacy. The concept of relative module privacy highlights a privacy level when an adversary obtains side-information that can compromise the existent privacy, which includes module privacy and (possibly) other privacy concepts. Examples of the other privacy concepts include objective privacy and differential privacy of which one or both may be enabled by the present disclosure.

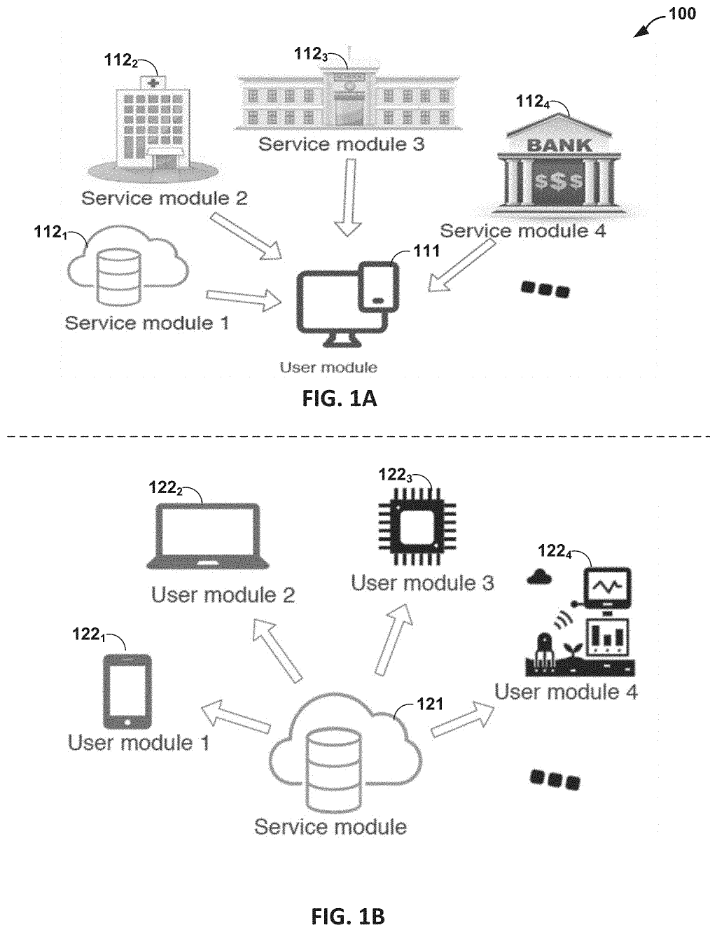

FIGS. 1A-B are block diagrams illustrating an example architecture 100 having at least two example operations for modules to exchange statistical information and build machine learning models, in accordance with one or more techniques of the disclosure. Example architecture 100 represents a decentralized network formed by multiple modules operating as peer learners.

FIG. 1A depicts an example operation where one entity system operates a user module 111 (“user module”) that receives assisted learning from entity systems operating, e.g., four service modules 112 (“service module 1”, “service module 2”, “service module 3”, and “service module 4”).

An entity system typically refers to one or more computing systems/networks that provide services to an entity's infrastructure (e.g., employees), including machine learning services via example architecture 100. In one example, an entity system may operate in example architecture 100 as either a service module or a user module, depending upon which operation is in effect. In general, a module represents one or more machine learning constructs.

A module generally represents a collection of machine learning resources. In one example, a module may include a (labeled) dataset {X₁; y₁} and a learner unit A₁ which applies a learning technique (e.g., a linear regression algorithm, a decision ensemble, a neural network, or another machine learning algorithm) to the labeled dataset {X₁; y₁} and produces a fitted dataset A₁ {X₁; y₁}. The labeled dataset {X₁; y₁} may include a set of observed labels that are either determined offline, provided by another learner unit on a remote device, determined via a machine learning model, or through another supervised learning algorithm. The labels represent the learning task of interest. The labels can be numerical responses in regression learning or numerically embedded class labels in classification learning. The learning technique applied by learner unit A₁ may create a function that processes, as input, the labeled dataset {X₁; y₁} and computes, as output, the fitted dataset A₁{X₁; \tilde{y}₁}. Over time, the learning technique applied by learner unit A₁ may update (e.g., train) the function to more accurately predict an expected label (e.g., value) from the feature set (X₁). To illustrate by way of example, an example learner unit function for a linear regression algorithm may be in the form of \tilde{y}₁=mX₁+b due to an expected linear distribution of the observed labels. Over time, the values for m and b are updated to more accurately predict the expected label \tilde{y}₁.

The module may further include a machine learning model that maps a feature set {X₁} of a data corpus to an observed label (y₁) denoting a particular value (e.g., regression) or classification. The model may be linear or non-linear in distribution. The model may be parameterized or non-parameterized. The module may further include a deterministic function that maps another labeled dataset (X₂; y₂) in the data corpus to a fitted labeled dataset using the learner unit A₁. The other labeled dataset (X₂;y₂) results from another learner unit A₂ from another entity system operating a module. Example architecture 100 may be a machine learning architecture that, over time, trains the learner unit (and/or the model) in each module.

The module, operating as either a user module or a service module as described herein, may desire assisted learning from another module in example architecture 100. The module may employ a number of techniques to select a proper module to exchange information. The following example technique can be used for a module to autonomously find one or more other modules to engage with for assisted learning: Before a module (Module 0) initializes an assisted learning with any other module (Module 1), Module 0 solicits from Module 1 a certain statistic calculated using Module 1's local data and based on that statistic, determines whether Module 1 is able to provide assistance. An example of such statistic is a linear combination of Module 1's feature variables, where the linear coefficients are randomly generated by Module 1 to properly privatize its locally held data. Upon receipt of the linearly combined variable, Module 0 will evaluate the statistical association between such a variable and its learning labels or fitted residuals calculated from its local data. Module 0 may use the calculated association to determine whether Module 1 has the potential to provide assistance.

As an alternative, the module may utilize a different technique to autonomously find one or more modules to engage with for assisted learning and that technique may be executed when the module employs a non-parametric machine learning model. If two (or more) modules are from a same data generating distribution (e.g., a centralized datasets of input features), then one module's learning unit and machine learning model should perform similarly when applied to another module's dataset. The module may use a certain statistic, such as a measurement of such similarity, to determine whether the module can be grouped with the other module of similar nature, and then, repeat a same determination for each other module. The module may identify one or more modules based on the certain statistics and further initialize an assisted learning procedure with either one other module or multiple other modules.

Regarding the above method, the module's learning unit and machine learning model include regression functions configured to, based on validation data, determine a (maximum) number of rounds of assistance in the assisted learning procedure with the other module. The validation data may be determined by cross validation within the other module(s).

In one example depicted in FIG. 1A, user module 111 may be a health insurance company that receives assisted learning from entity systems operating as service modules 112. As illustrated, the health insurance company, as user module 111, may receive assisted learning from service modules 112 including a generic service module, a hospital, a school, and a bank, respectively. The generic service module represents another possible entity system including another health insurance company. When the health insurance company requests assisted learning from the generic service module, the hospital, the school, and/or the bank, the health insurance company in response, receives statistical information to improve upon accuracy of the (observed) labeled dataset {X₁; y₁} and the learner unit A₁.

The health insurance company, the generic service module, the hospital, the school, and/or the bank collect various information for different feature sets from a substantial number of people. The bank may store attributes for features, such as deposit, salary, debt, and/or the like while the health insurance company stores feature attributes in various types of medical records. If the health insurance company wants to develop a new insurance product with high return, it is beneficial to know the financial status of each targeted client. Yet, the bank will not directly disclosure any individual-level data even if they are perturbed. There exists an incentive to both parties that the bank provides services that do not directly transmit data but still provide relevant information for the health insurance company to facilitate its own learning.

To provide an enhanced level or privacy, the health insurance company receives certain statistical information with the generic service module, the hospital, the school, and/or the bank. By exchanging the certain statistical information, the generic service module, the hospital, the school, and/or the bank may retain sensitive data in secure data stores. Hence, the bank in the above-mentioned example does not disclose any individual-level data such as a financial status to the health insurance company. The bank also does not expose their proprietary learner unit A_{bank} (e.g., a machine learning model), or any information associated with their proprietary learner unit A_{bank}. This may include the bank's proprietary feature set, a model used in mapping the feature set (X_{bank}) to a label set (y_{bank}), and a learning technique to fit the label set (y_{bank}) to a fitted label set (\tilde{y}_{bank}).

Therefore, by implementing the techniques described herein, the health insurance company, operating as the user module 111 in FIG. 1A, may use the certain statistical information to improve upon the company's propriety learner unit A_{Insurance} while neither disclosing any proprietary information (e.g., an observed label set) nor receiving any feature information or model information from any of the service modules 112 in FIG. 1B. Once the learner unit A_{Insurance} is sufficiently trained, the health insurance company may use the learner unit A_{Insurance}, to make predictions regarding existing users and new users. In one example, the health insurance company may query service modules 112 for set of predictions on a same user and, combining the set of predictions with a local prediction regarding the same user, produce a predicted label for that same user.

The nature of the certain statistical information may depend upon which learning technique is employed by an entity system, such as the health insurance company when operating as user module 111. User module 111 may be configured with a corresponding model for any learning technique (e.g., linear regression) and, by way of assisted learning, receive statistics related to a compatible model in one or more service modules 112. User module 111 may employ a number of statistical method to update the corresponding model with the received statistics. In one example, if the health insurance company is creating a learner unit using any example learning technique and a corresponding model, appropriate statistical information may include one or more residuals from fitting a label set into a fitted label set when the fitted label set and possibly the label set are based upon a feature set. The example learning technique may update the learner unit (e.g., the corresponding model) to better approximate the fitted label set from the same feature set.

FIG. 1B introduces a different perspective into example architecture 100 from FIG. 1A: An entity system, operating a generic service module 121, engages in assisted learning with, e.g., four entity systems that operate user modules 122. In general, user modules 122₁ . . . 122₃ (collectively referred to as “user modules 122”) and generic user module 122₄ form an assisted learning framework where multiple organizations with discordant learning goals/objectives and heterogeneous/multimodal data whose sharing is prohibited. Over a number of iterations of assisted learning, generic service module 121 and each of user modules 122 limit their data exchanges to task-relevant statistics instead of raw data.

In one example, generic service module 121 (e.g., a clinic research laboratory) provides other entity systems, including the four entity systems that operate user modules 122, with various services (e.g., clinical research services) without sharing sensitive data (e.g., patient data) and may employ artificial intelligence (e.g., machine learning models) in these services. To provide the four entity systems that operate user modules 122 with assisted learning, generic service module 121 may share statistical information corresponding to a machine learning model.

In one example, user module 122₁ (e.g., a computing device in a hospital) and generic service module 121 (e.g., a clinic research laboratory) both store feature sets from a same group of people and use those features in separate models. Both generic service module 121 and user module 122₁ use their respective models to predict a random hospital patient's Length of Stay (LOS), which is one of the most important driving forces of hospital costs. While user module 122₁ trains its proprietary model, generic service module 121 provides statistical information that user module 122₁ utilizes to advance the proprietary model's training.

In a multi-agent example, another user module, user module 122₂ (e.g., a computing device in a health insurance company) may also receive assisted learning in the form of statistical information from generic service module 121. Because user module 122₂ builds its own proprietary model, that model's parameters and feature sets may differ from the models of the generic service module 121 and user module 122₁. Furthermore, generic service module 121 may provide user module 122₂ with different statistical information. In some examples, user module 122₂ trains the proprietary model with a different objective than the models of the generic service module 121 and user module 122₁, such as a prediction other than the random patient's LOS. Even if user module 122₂ trains the proprietary model with the same objective of predicting the random patient's LOS, the model's prediction may be different from the model of user module 122₁.

In any of the above examples, user module 122₁ and/or user module 122₂ may send their own respective task-related statistics to generic service module 121 and in turn, receive generic service module 121's task-related statistical response based on each user module's respective task-related statistics. Each module generates task-related statistics that do not expose any of that module's (e.g., proprietary) feature data (e.g., patient data) nor label data (e.g., model prediction data). In this manner, each module maintains the privacy of their confidential data (e.g., differential privacy) as well as their proprietary model (e.g., module privacy). In some instances, a given module maintains objective privacy as well by not transmitting any data indicating the given module's proprietary model's prediction.

In general, generic service module 121 may create, train, and/or deploy a machine learning model having a supervise relation (e.g., a mapping) between a specific set of input features (e.g., a feature set X) and an output prediction (e.g., a label set Y). In another example, generic service module 121 and one or more user modules 122 may build models configured to predict a certain health index for the random patient. Generic service module 121 may create a learner unit A to train a supervise function f to fit the random patient's health index such that the function f may better predict for that patient a revised health index given a different set of features. With respect to user module 122₁ (e.g., a doctor's computing device in a hospital) which provides services (e.g., health services of which some employ artificial intelligence such as machine learning models) regarding the above patient, these services may rely upon an accurate machine learning model for a representative learner unit, learner unit A₁.

In one example, generic service module 121 determines parameters (e.g., weights) for the mathematical function f that processes, as input, the feature set X and generates, as output, the label set Y. The label set Y may be a fitted label set such that each fitted label is an expected outcome (e.g., expected health index) in accordance with a distribution of the mathematical function f. During training, a set of residuals between the fitted label set and an observed label set (e.g., observed health indexes) are used to update the function f to more accurately predict the expected outcome.

Furthermore, a second set of residuals between a second set of fitted labels and the set of residuals (as the observed label set) are used to update the function fin the machine learning model for the learner unit A. The hospital operating as user module 122₁ may include a learner unit A₂ and a machine learning model relating another feature set (X₂) with the certain health index to produce the second fitted label set (Y₂). User module 122₁ may determine the second set of residuals between the second fitted label set (Y₂) and the set of residuals from the generic service module 121. A different hospital operating as user module 122₂ may include a learner unit A₃ and a machine learning model relating another feature set (X₃) with the health index to produce yet another fitted label set (Y₃). The generic service module 121 may use another set of residuals between label set Y₃ and the set of results to update the mathematical function f for learner unit A₁. Each user module includes a feature set that contains different (or partially overlapping) features that correspond to the same group of patients.

It should be noted that the above-mentioned health index differs from a matrix index or column vectors index. Each module maintains input feature sets in a matrix or as column vectors where each column is a feature vector for all patients and each row is a single patient's feature set. Two or more modules have collated matrices/column vectors if their rows are aligned with a common index, such as a timestamp, a username, or a unique identifier.

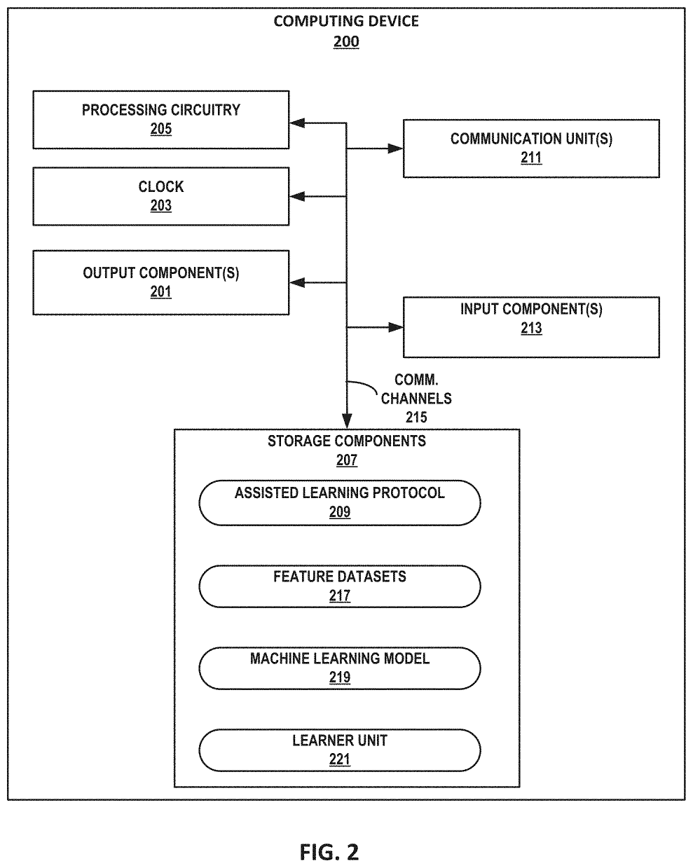

FIG. 2 is a block diagram illustrating example computing device 200 within an entity system for example architecture 100 of FIG. 1A and/or 1B, in accordance with one or more techniques of the disclosure. Computing device 200 of FIG. 2 is described below as an example computing device being used by an entity system while operating as either a user module or a service module of FIG. 1A and/or 1B. FIG. 2 illustrates only one example of computing device 200, and many other examples of computing device 200 may be used in other instances and may include a subset of the components included in example computing device 200 or may include additional components not shown in example computing device 200 of FIG. 2.

As shown in the example of FIG. 2, computing device 200 includes one or more output components 201, clock 203, processing circuitry 205, one or more storage components 207, one or more communication units 211, and one or more input components 213. Communication channels 215 may interconnect each of the components 201, 203, 205, 207, 211, and 213 for inter-component communications (physically, communicatively, and/or operatively). In some examples, communication channels 215 may include a system bus, a network connection, an inter-process communication data structure, or any other method for communicating data.

One or more communication units 211 of computing device 200 may communicate with external devices, such another of computing devices 102 of FIG. 1A and/or FIG. 1B, via one or more wired and/or wireless networks by transmitting and/or receiving network signals on the one or more networks. Examples of communication units 211 include a network interface card (e.g. such as an Ethernet card), an optical transceiver, a radio frequency transceiver, a GPS receiver, or any other type of device that can send and/or receive information. Other examples of communication units 211 may include short wave radios, cellular data radios, wireless network radios, as well as universal serial bus (USB) controllers.

One or more input components 213 of computing device 200 may receive input. Examples of input are tactile, audio, and video input. Input components 213 of computing device 200, in one example, includes a presence-sensitive input device (e.g., a touch sensitive screen, a PSD), mouse, keyboard, voice responsive system, video camera, microphone or any other type of device for detecting input from a human or machine. In some examples, input components 213 may include one or more sensor components one or more location sensors (GPS components, Wi-Fi components, cellular components), one or more temperature sensors, one or more movement sensors (e.g., accelerometers, gyros), one or more pressure sensors (e.g., barometer), one or more ambient light sensors, and one or more other sensors (e.g., microphone, camera, infrared proximity sensor, hygrometer, and the like).

One or more output components 201 of computing device 200 may generate output. Examples of output are tactile, audio, and video output. Output components 201 of computing device 200, in one example, includes a PSD, sound card, video graphics adapter card, speaker, cathode ray tube (CRT) monitor, liquid crystal display (LCD), or any other type of device for generating output to a human or machine.

Clock 203 is a device that allows computing device 200 to measure the passage of time (e.g., track system time). Clock 203 typically operates at a set frequency and measures a number of ticks that have transpired since some arbitrary starting date. Clock 203 may be implemented in hardware or software.

Processing circuitry 205 may implement functionality and/or execute instructions associated with computing device 200. Examples of processing circuitry 205 include application processors, display controllers, auxiliary processors, one or more sensor hubs, and any other hardware configure to function as a processor, a processing unit, or a processing device. Assisted learning protocol 209 may be operable by processing circuitry 205 to perform various actions, operations, or functions of computing device 200. For example, processing circuitry 205 of computing device 200 may retrieve and execute instructions stored by storage components 207 that cause processing circuitry 205 to perform the operations of assisted learning protocol 209. The instructions, when executed by processing circuitry 205, may cause computing device 200 to store information within storage components 207.

One or more storage components 207 within computing device 200 may store information for processing during operation of computing device 200 (e.g., computing device 200 may store data accessed by assisted learning protocol 209 during execution at computing device 200). In some examples, storage component 207 includes a temporary memory, meaning that a primary purpose of one example of storage components 207 is not long-term storage. Storage components 207 on computing device 200 may be configured for short-term storage of information as volatile memory and therefore not retain stored contents if powered off. Examples of volatile memories include random-access memories (RAM), dynamic random-access memories (DRAM), static random-access memories (SRAM), and other forms of volatile memories known in the art.

Storage components 207, in some examples, also include one or more computer-readable storage media. Storage components 207 in some examples include one or more non-transitory computer-readable storage mediums. Storage components 207 may be configured to store larger amounts of information than typically stored by volatile memory. Storage components 207 may further be configured for long-term storage of information as non-volatile memory space and retain information after power on/off cycles. Examples of non-volatile memories include magnetic hard discs, optical discs, floppy discs, flash memories, or forms of electrically programmable memories (EPROM) or electrically erasable and programmable (EEPROM) memories. Storage components 207 may store program instructions and/or information (e.g., data) associated with assisted learning protocol 209. Storage components 207 may include a memory configured to store data or other information associated with assisted learning protocol 209.

Assisted learning protocol 209 connects learner unit 221 of an entity system to example architecture 100 to operate as a service module, a user module, or both user module and service module. As a service module, assisted learning protocol 209 provides user modules with a service (e.g., an artificial intelligence service); as a user module, assisted learning protocol 209 requests services from service modules. The entity system, as described herein, may include a number of computing devices, such as computing device 200, for use in creating, training, and deploying machine learning constructs (e.g., models). The entity system may provide these computing devices to example architecture 100 to run as modules (e.g., user modules, service modules, or both user modules and service modules).

In either capacity, an example computing device exchanges with other computing devices machine learning information to improve upon a modeling of user data. In some examples, assisted learning protocol 209 distributes to one or more computing devices in example architecture 100 statistical information for improving each computing device's learner unit and any machine learning model used by that learner unit. Assisted learning protocol 209 may perform such distribution in response to receiving statistical information from another computing device. Assisted learning protocol 209 may use the received statistical information to improve learner unit 221 and any machine learning model 219 used by learner unit 221.

One operation of assisted learning protocol 209 is to improve a learning quality of at least learner unit 221, by allowing computing device 200, operating as a module, to exchange statistics with other computing devices operating as modules. In one example, for computing device 200 to receive assistance from other modules in example architecture 100, feature datasets 217 and respective feature datasets from the other modules are to be aligned or partially aligned (e.g., collated). Two datasets D1 and D2 are aligned datasets if the two datasets can be aligned by some common feature (referred to as index). For example, the common index can be a date. Having aligned or partially aligned feature datasets, assisted learning protocol 209 may further improve upon a learning quality of learner unit 221.

One example technique to improve the machine learning capabilities of computing device 200 directs assisted learning protocol 209 to create, by processing circuitry 205, learner unit 221 by fitting, into a first fitted label set, an initial label set using at least one first learning technique and a first feature set. In accordance with the example technique, the assisted learning protocol 209 proceeds to send, by the processing circuitry 205, to at least one module in a machine learning architecture, first statistical information defined by at least one residual from fitting the label set into the first fitted label set, wherein the at least one assisting module is operative to fit, into a second fitted label set, the first statistical information using at least one second learning technique and a second feature set. Assisted learning protocol 209 receives, by the processing circuitry 205, second statistical information from the at least module, the second statistical information being defined by at least one residual from fitting the second label set. The example technique prompts assisted learning protocol 209 to update, by the processing circuitry 205, the learner unit 221 by fitting, into a third fitted label set, the second statistical information using the at least one first learning technique.

Learner unit 221, as a component of computing device 200, may represent logic implementing computational functionality or processor-executable instructions. Via assisted learning protocol 209, computing device 200 trains machine learning model 219 for use by learner unit 221, for example, in generating predictions. In one example, machine learning model 219 may include a linear distribution relating a feature set X to a label Y and learner unit 221 may fit label Y along the same linear distribution and produce a fitted label \tilde{Y}. In another example, while machine learning model 219 may include a non-linear distribution relating a feature set X to a label Y, learner unit 221 may include a function to fit label Y along a linear distribution and produce a fitted label \tilde{Y}. The function in learner unit 221 may approximate the label Y more efficiently than machine learning model 219.

In one particular example, computing device 200 represents a hospital device configured with a set of labeled data (X0; Y0) and supervised learning algorithms for performing machine learning services for hospital patients and/or personnel. The hospital may be an organization with a number of divisions, and for the hospital, computing device 200 directs assisted learning protocol 209 with m divisions (e.g., Intensive Care Unit, In-hospital laboratory, out-patient laboratory, and/or the like) performing different learning tasks with distinct data (Xᵢ;Yᵢ) where i=1,2, . . . ,m and learning models, where (Xᵢ) for i=1,2, . . . , m can be collated. The hospital desires assistance from others to facilitate training for its model while retaining its sensitive data and for potential rewards, may assist others in the training of their model with their own learning algorithm. Because the m divisions share a substantial portion of the same sensitive data, there m divisions may run off centralized datasets. However, if there is a substantial risk to sharing any sensitive data between them, the partially aligned or aligned data sets are on remote devices. An example learning algorithm may represent a linear regression, a decision ensemble, a neural network, or a set of models from which a suitable one is chosen using model selection techniques. For example, when the least squares method is used to learn the supervised relation between X and y, then a prediction function is a linear operator for a predictor feature.

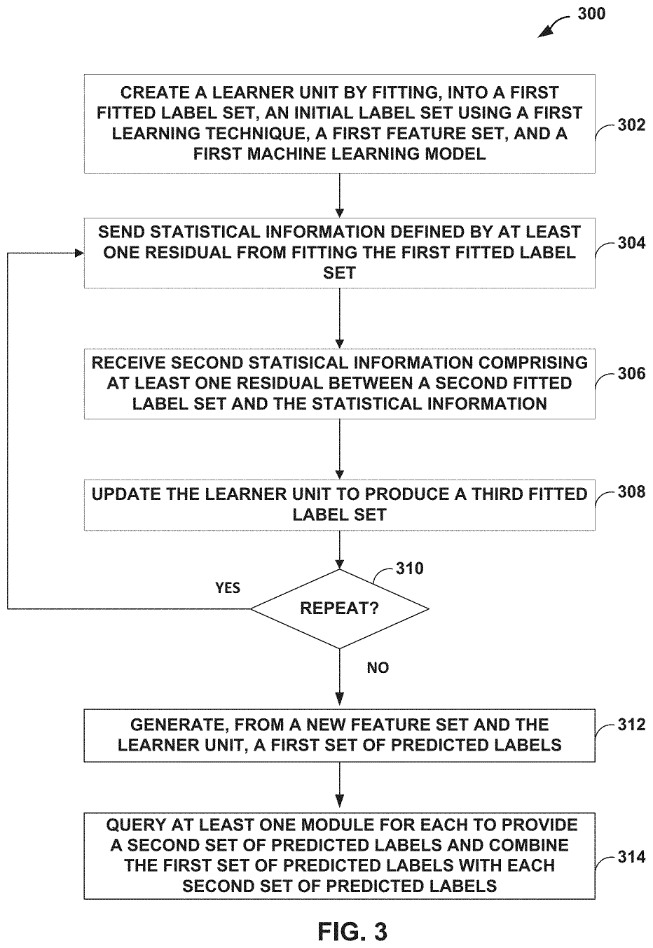

FIG. 3 is a flowchart illustrating an example process 300 for providing assisted learning to a computing device of an example architecture 100 of FIG. 1A and/or FIG. 1B, in accordance with one or more techniques of the disclosure. For purposes of illustration only, FIG. 3 is described with respect to FIG. 2.

In computing device 200 operating as a module in example architecture 100, processing circuitry 205 creates a learner unit (e.g., learner unit 221 of FIG. 2) by fitting, into a first fitted label set, an initial label set using a first learning technique, a machine learning model, and a first feature set (302). In general, the initial label set may be generated by any module desiring assisted learning, which, in some instances, may be computing device 200 operating as a user module or a service module. In some examples, computing device 200 employs a mechanism such as machine learning model 219 to determine the initial label set from the first feature set. In other examples, the initial label set may be provided by alternative means, such as another module in example architecture 100 (e.g., a user module providing labels in a query).

In some examples, machine learning model 219 includes a technique (e.g., a mathematical function or method) that processes, as input, the first feature set and produces, as output, a set of expected labels to be used as the initial label set. In some examples, the technique of machine learning model 219 codifies a relationship between one or more feature attributes of each user in the first feature set and a particular label (e.g., a regression label) indicating some knowledge. Using the technique of machine learning model 219, processing circuitry 205 creates learner unit 221 by determining a function ‘f’ configured to fit the initial label set into the first fitted label set. Following the first learning technique, learner unit 221 may fit the function ‘f’ by tuning terms (e.g., parameters or hyper-parameters) of the function ‘f’ until the first fitted label set closely approximates the initial label set. In some examples, learner unit 221 generates the function ‘f’ to have a linear relationship between the first feature set and the initial label set.

To illustrate by way of example, in a linear regression learning technique as the first learning technique, function ‘f’ follows a linear distribution. Each fitted label may be considered an expected data point and each initial label may be considered an observed data point such that a set of residuals between expected and observed data points can be used to update (e.g., fit) the function ‘f’ in learner unit 221. In some examples, parameters (e.g., weights, constants, etc.) of function ‘f’ may be adjusted (e.g., tuned) to fit the linear distribution to the initial label set. Each residual may be used in the example process 300 as statistical information to be exchanged with one or more modules. The example process 300 may limit assisted learning protocol 209 to ‘m’ modules for exchanging statistical information. In one example, based on communication bandwidth, cost constraints, and/or computational overhead, assisted learning protocol 209 may only select a subset of ‘m’ modules to exchange statistical information.

Processing circuitry 205 sends, to another computing device, first statistical information defining at least one residual between the first fitted label set and the initial label set (304). In some examples, processing circuitry 205 sends the first statistical information to a remote computing device operating as a module in example architecture 100, and the remote computing device, in turn, uses the at least one residual as an observed label set and computes another set of residuals with a second fitted label set. Similar to computing device 200, the remote computing device may use a machine learning model to determine, from a second feature set, a second set of labels. The remote computing device may employ a learner unit to determine, from the second set of labels, the second fitted label set using the second learning technique. In some examples, the remote device updates the learner unit to better fit the at least one residual. The remote computing device communicates to computing device 200 the other set of residuals as second statistical information.

Processing circuitry 205 receives the second statistical information comprising at least one second residual between the second fitted label set and the first statistical information (306). The computing device 200 may consider the at least one second residual in the second statistical information to be observed labels. Based upon the mathematical function ‘f’ in learner unit 221, processing circuitry 205 determines the third fitted label set using the (local) first feature set and determines a third set of residuals between the fitted label set and the observed labels. In some examples, prior to determining the third fitted label set, the learner unit 221 updates function ‘f’ to fit the at least one second residual, for instance, by tuning coefficients, constants, or other components of the function ‘f’ to include the at least one second residual in the relationship (e.g., the linear relationship) between feature attributes of the first feature set and a label space. In operation, the at least one second residual is a projection onto the label space (e.g., column space) of the first feature set. Processing circuitry 205 updates the learner unit 221 to produce a third fitted label set based upon the second statistical information (308). Updating the learner unit 221 causes an update to the function ‘f’ and (perhaps) the machine learning model 219. The third fitted label set is more accurate than the first fitted label set by taking into account the residuals in the second statistical information.

Processing circuitry 205 repeats the steps of sending of statistical information, receiving of the second statistical information and updating of the learner unit 221 (e.g., a training stage) for a number of iterations (310). For example, processing circuitry 205 may send third statistical information defined by at least one third residual from fitting the third fitted label set and a corresponding machine learning model. A third particular residual may be determined based on (e.g., comparing) a first third fitted label of the third fitted label and at least one of a second particular residual of the at least one second residual, the first initial label of the initial label set, or the first observed data (e.g., a first observed label) in the first feature set.

During the training stage, processing circuitry 205 may update the function ‘f’ and/or the machine learning model 219 for the learner unit 221 to better fit any received statistical information. In one example, the number of iterations can be limited based upon an information set amongst all modules (including computing device 200 and any remote computing device). In one example, processing circuitry 205 repeats the sending and the receiving until an out-sample error no longer decreases.

After the number of iterations has elapsed, processing circuitry 205 proceeds to a prediction stage, indicating to the machine learning architecture that the learner unit 221 is sufficiently trained and deployable as either a service module or a user module in the machine learning architecture. During this stage, processing circuitry 205 of computing device 200 provides various services in response to requests from entity systems operating as user modules. Processing circuitry 205 uses learner unit 221 to predict a set of labels based upon new feature datasets. In one example, processing circuitry 205 generates, from a new feature set and the learner unit 221, a first set of predicted labels (312). The new feature set may include one or more input features (e.g., predictors) for a new person (e.g., a user or a patient) such that when the processing circuitry 205 applies each machine learning model of learner unit 221 to the new feature set, processing circuitry 205 generates the first set of predicted labels. For example, processing circuitry 205 may apply corresponding machine learning models for the first fitted label set and third fitted label set to the new feature set and each model may generate a first label and a second label to be combined into the first set of prediction labels. Then, processing circuitry 205 queries at least one remote computing device operating the at least one module and obtains, as a response from each module, a second set of predicted labels for the new feature set (314). An example module may apply corresponding second machine learning models for the second fitted label set and a fourth fitted label set where the fourth fitted label set may be produced by fitting, into the fourth fitted label set, the at least one third residual. To complete the prediction, processing circuitry 205 combines the first set of predicted labels and the second set of predicted labels into a final set of predicted labels.

The above can be contextualized with the following examples. The above computing device may be an intensive care unit (ICU) at a hospital and is developing a module to predict the length of in-hospital stay, using its collected patient data. The ICU employs learner unit 221 to benefit from diverse information sources including other in-patient/out-patient entities, such as a pharmacy or a laboratory. The ICU and at least one of these entities form a portion of machine learning architecture 100 and have many overlapping patients that can be collated by identifiers (e.g., email and username). If the pharmacy provides the ICU with assisted learning, both entities may utilize separate feature sets from decentralized datasets; however, neither the ICU nor the pharmacy will share their private data and models. This may be true even if the hospital and the pharmacy are a part of a single organization (e.g., as divisions), use centralized datasets, and/or similar features. They may use the assisted learning protocol 209 of so that the pharmacy can assist ‘the ICU can improve its predictive accuracy.

Procedure 1 (reprinted below) illustrates an example implementation of an assisted learning protocol between module M₀ and m other modules. In the training stage (e.g., training process 300 of FIG. 3), at each round k, module M₀ first sends a query to each module Mⱼ by transmitting statistic information e_{j,k}. Upon receipt of the query, module Mⱼ treats e_{j,k} as labels and fits a learner unit Â (based on the data aligned with such labels) into fitted labels \tilde{e}_{j,k}, which are sent back to module M₀. Module M₀ processes the collected responses \tilde{e}_{j,k}, . . . (j=1, . . . , m), and initializes the k+1 round of communications. After the above procedure stops at an appropriate stopping time k=K, the training stage for module M0 is suspended. In the prediction stage, upon arrival of a new feature vector x*, user 0 queries the prediction results Â_{j,k} (xⱼ^{*}) (k=1, 2, . . . , K) from module j, where xⱼ^{*} denotes the component of x* observed by module j, and combines them to form the final prediction \tilde{y}*.

Procedure 2 (reprinted below) illustrates another example implementation of an assisted learning protocol between module 0 and another module 1. In the first round of the training stage, module 0 fits label set y into fitted label set \tilde{y} using A₀ but only sends fitted residuals e₁ to module 1. Module 1 considers the fitted residuals e₁ as an observed label set and fits residuals e₁ using learner unit A₁ and local feature datasets into a fitted label set \tilde{e}₁. Then, instead of sending the learner unit A₁ or any feature datasets, Module 1 sends fitted residuals \tilde{e}₁ back to module 0. Module 0 then initializes the second round by treating the fitted label set \tilde{e}₁ as the same as the observed label set y in the first round. This exchange of statistics repeats until the out-sample error (as measured by, e.g. cross-validation) of module 0 satisfies one or more criterion (e.g., falls below a threshold or plateaus by no longer decreasing). In the prediction stage, for a new object, user 0 queries the prediction results Â_{k,1}(xⱼ^{*}) (k=1, 2, . . . , K) from module 1, and form the final prediction \tilde{y}*=Σ_{j=0,1}Σₖ₌₁^{K}Â_{j,k}(xⱼ^{*}).

Procedure 2
Assisted Training Stage
Input: Two modules: Module 0 with task label y ∈ Rⁿ and local data X₀,
Module 1 with local data X₁ that provides assistance
Initialization: eₖ = y, round k = 1
For k = 1, . . . , K:
Module 0 fits a supervised learning model using (eₖ, X₀) as labeled data
Module 0 records its fitted model Â_{0,k}, calculates the residual rₖ, and
sends rₖ to Module 1
Module 1 fits a supervised model using (rₖ, X₁) as labeled data
Module 1 records its fitted model Â_{1,k}, calculates the residual \tilde{e}ₖ, and
sends \tilde{e}ₖ to Module 0
Module 0 initializes the k + 1 round by setting eₖ₊₁ = \tilde{e}ₖ
Output: Module i's local models Â_{i,k}, i = 0, 1, k = 1, . . . , K
Assisted Prediction Stage
Input: new data x*, whose components x₀^{*} is observed by Module 0 and
another component x₁^{*} is observed by Module 1
Module 0 queries the prediction results from Module 1's local models:
\tilde{y}_{1,k}^{*} = Â_{1,k}(x₁^{*}) for k = 1, . . . , K
Module 0 also calculates the prediction from its local models: \tilde{y}_{0,k}^{*} =
Â_{0,k} (x₀^{*}) for k = 1, . . . , K
Module 0 forms the final prediction \tilde{y}* = Σ_{1≤k≤K}(\tilde{y}_{0,k}^{*} + \tilde{y}_{1,k}^{*})
Output: Assisted prediction \tilde{y}*

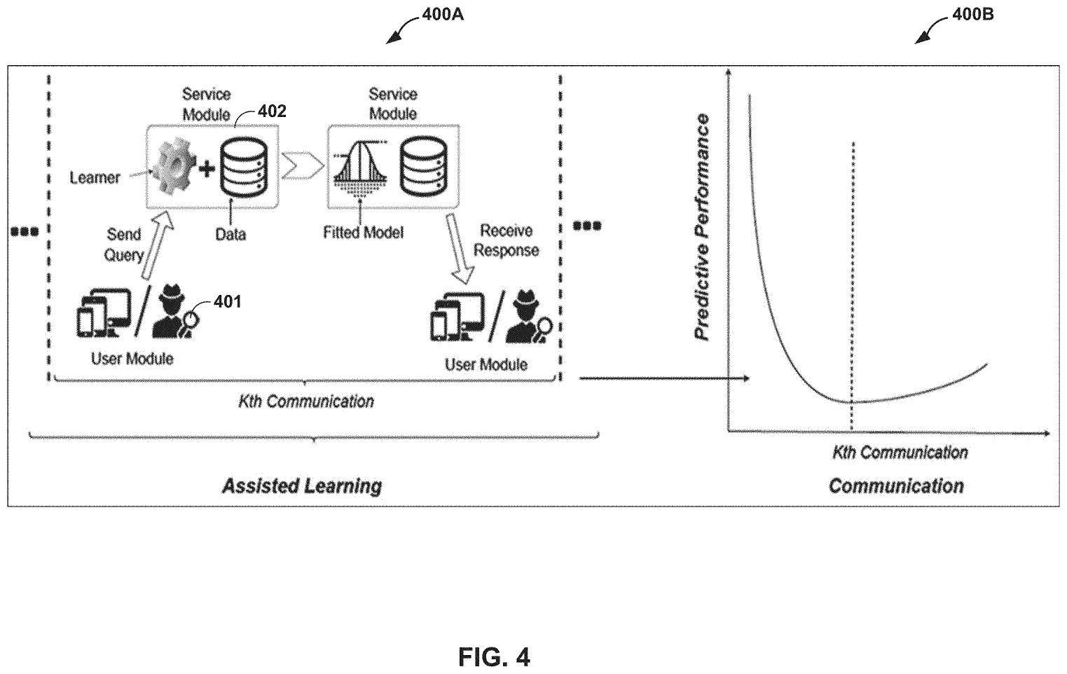

FIG. 4 is a block diagram illustrating a relationship between assisted learning and predictive performance as achieved by an example architecture 100 of FIG. 1A and/or FIG. 1B, in accordance with one or more techniques of the disclosure. Left plot 400A of FIG. 4 depicts a single round of assisted learning protocol between modules, user module 401 and service module 402, while the right plot 400B depicts k rounds of assisted learning as defined by K rounds of communications between these modules.

Right plot 400B highlights a stopping criterion for communications between modules during the assisted training process. While more rounds of communications typically to bring more information exchange and better fitting to the data, excessive communications often bring overfitting so that the actual out-sample predictive performance of the module being assisted actually becomes worse.

Computing an out-sample loss from the candidate methods and the pulled data of all modules (including an originating module) may be used to determine a number of communications for the assisted learning protocol. This quantity provides a theoretical limit or benchmark on what the assisted learning protocol described herein can bring to a computing device operating as a module. Techniques for computing the out-sample loss can be found in (e.g., Section 4.3 of) non-patent literature entitled, “Assisted Learning: A Framework for Multi-Organization Learning,” which has been incorporated by reference in its entirety.

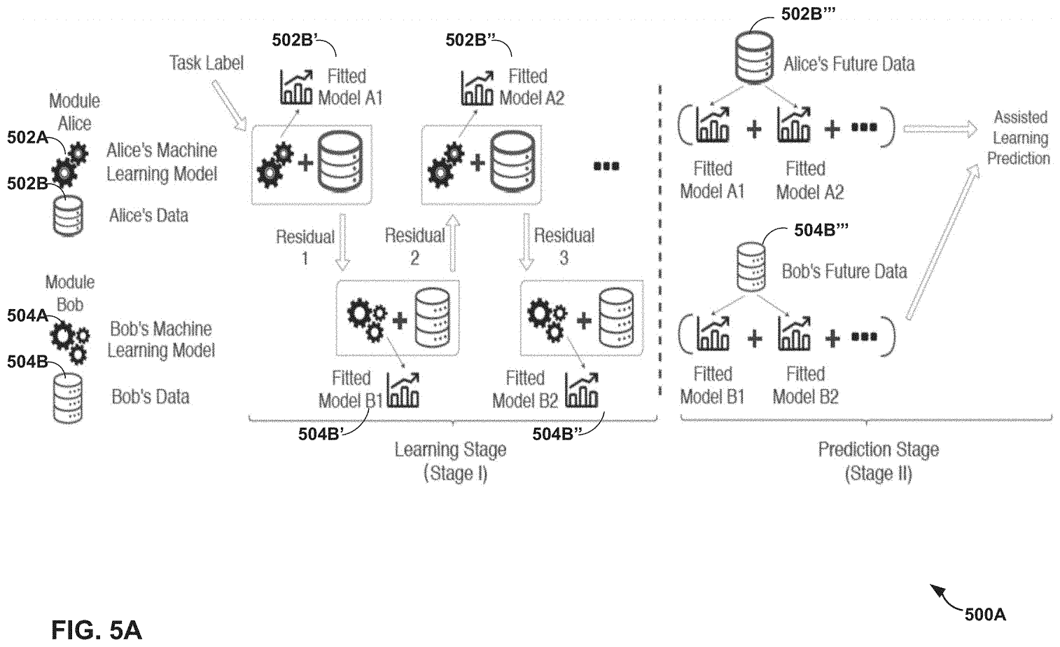

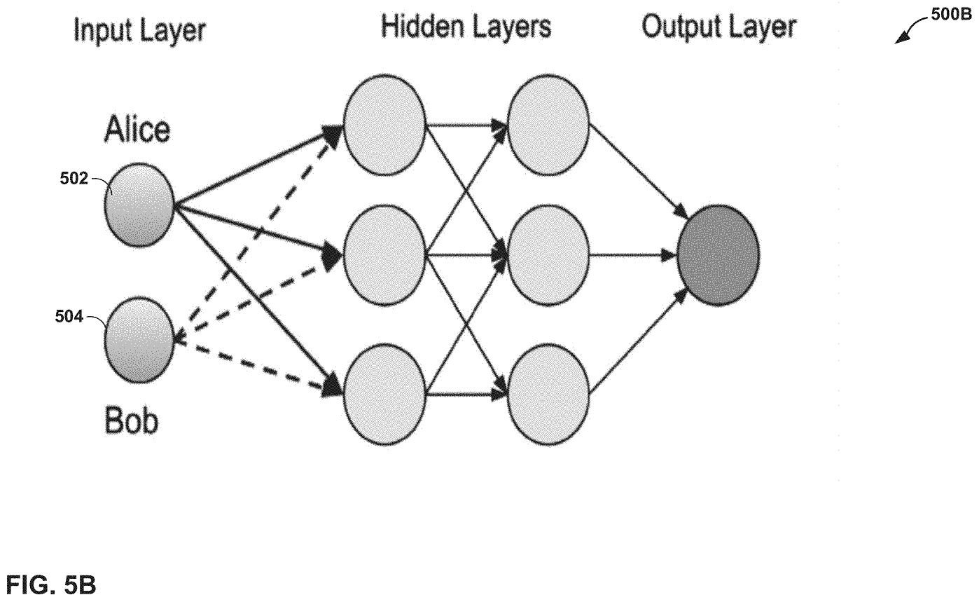

FIG. 5A is an illustration of assisted learning protocol 500A and FIG. 5B is an illustration of model 500B generate by assisted learning protocol 500A of FIG. 5A, in accordance with one or more techniques of the disclosure.

As depicted, assisted learning protocol 500A includes a learning stage (i.e., a learning or training process) and a prediction stage (i.e., a prediction process) for both Alice 502 and Bob 504. Alice 502 and Bob 504 represent modules with learner units configured to participate in assisted learning protocol 500A. As described herein, each module 502, 504 includes separate datasets 502A and 504A that is partially aligned (e.g., collated) and separate, private models 502B and 504B.

Although model 500B is configured to be a feedforward neural network, any other machine learning construct may be implemented instead in the context of assisted learning protocol 500A. Model 500B is illustrated (for brevity reasons) as a three-layer feedforward neural network with Alice 502's weights w_{a,k} (denoted by solid lines) and Bob 504's weights w_{b,k} (denoted by dash lines). Both sets of weights are input-layer weights at the kth round of assistance for Alice and Bob, respectively. Other weights (if any) are denoted at the kth round of assistance by wₖ. If X_{A} and X_{B} represent observed datasets for Alice 502 and Bob 504, w_{a,k}X_{A} and w_{b,k}X_{B} represent residuals from Alice's model 502B and Bob's model 504B.

In the learning stage for assisted learning protocol 500A, at a first round of k rounds of assistance, Alice 502 fits model 502B into model 502B′ with an initial label (observed) for a feature vector V_{a,i} by training model 502B to predict the initial label. At the end of training and when a first fitted label for the feature vector V_{a,i} is an acceptable and approximate prediction of the initial label, Alice 502 produces model 502B′ (e.g., to include a first fitted label set). When any label is associated with an objective, regarding of it be a public one or a private one, may be referred to as a task label. Alice 502 sends a query to Bob 504 including latest statistics, including residual 1 (e.g., a first residual), based on the first fitted (task) label determined from model 502B′ for the feature vector V_{a,i} in datasets 502A. As described herein, residual 1 may be the first residual value between the initial label value and the predicted task label.

In the context of a LOS prediction task, an example for residual 1 may be a difference between a patient's actual length of stay denoted by the initial label value and a predicted length of stay in a hospital denoted by the first fitted label value determined by model 502B′. When Alice 502 employs model 500B, Alice 502's weights w_{a,k} are used to estimate the patient's predicted length of stay. In some examples, expression w_{a,k}X_{A} results in the example residual 1 if observed data X_{A} includes the initial task label (which in this case represents the patient's actual or observed length of stay) in the feature vector V_{a,i}. For other patients' feature vectors, these vectors have to aligned on a same task label. Hence, the first fitted label is computed from other vector values to represent the patient's predicted length of stay. Alice 502 proceeds to produce w_{a,k}X_{A} as a value for the example residual 1 and if k is even, Alice 502 updates weights w_{a,k} into weights w_{a,k+1} using backpropagation but if k is odd, Alice 502 sets weights w_{a,k+1} for next round with weights w_{a,k}.

Upon receipt of the query, Bob 504 treats residual 1 as a label (e.g., observed task label) and fits model 504B (based on observed datasets 504A which is aligned with first residuals as labels) to generate fitted model 504B′. This may be accomplished by training model 504B with residual 1 as the label for the feature vector V_{a,i} until a stop criterion is met; as a result, fitted model 504B′ is configured to predict residual 1 with a second fitted label. By further training model 502 and model 504 with each other's statistical information, each of fitted models 502B″ and 504B″ is trained using the other's model's configuration. Bob 504 determines a value for residual 2 by comparing the second fitted label and residual 1 and computing a difference value between residuals. Bob 504 then sets residual 2 as the difference value and sends residual 2 to Alice 502. When Bob 504 employs model 500B, Bob 504 produces w_{b,k}X_{B} as residual value between the patient's observed length of stay and the patient's predicted length of stay using Bob 504's feature vector x_{B} and model 504B. To determine the above residual, Bob 504's weights w_{b,k} and model 504B are first fitted to predict w_{a,k}X_{A} for residual 1, and then, used to generate a residual label (prediction). Bob 504 proceeds to produce w_{b,k}X_{B} as a residual value between the patient's observed length of stay and Bob 504's predicted value. If k is odd, Bob 504 updates weights w_{b,k} into weights w_{b,k+1} using backpropagation.

In the context of LOS prediction, fitted model 504B′ results from modifying model 504B to predict residual 1 based on a different feature vector for the same patient. Fitted model 504B′ is used to generate a residual label for residual 1, which is compared to residual 1 and the difference is used to determine residual 2. Fitted model 502B′ generates residual 1 to represent the residual between the patient's actual length of stay and the predicted length of stay based on model 502B′ and the residual label indicates a predicted residual between the patient's actual length of stay and a predicted length of stay based on model 504B. Residual 2, therefore, is an actual residual between the predicted residual and residual 1. Residual 3, therefore, is an actual residual between a predicted residual for residual 2 and residual 2. Alice 502 processes Bob 504's response and fits fitted model 502B′ to generate fitted model 502B″. Alice 502 treats residual 2 as a label (e.g., an initial label and/or observed data) for model 502B′ and trains (e.g., modifies) that model into a new fitted model (e.g., fitted label set) configured to predict residual 2.

Alice 502 prepares for a next round of learning/training by determining residual 3 by comparing residual 2 to a residual label generated by fitted model 502B″ where the residual label is a predicted value for residual 2. A difference between the fitted residual label and residual 2 and becomes residual 3. In the next round, iteration k+1, Bob 504 receives residual 3 and uses that value as a label for fitted model 504″. Bob 504 proceeds to fit fitted model 504″ to residual 3 in a manner similar to residual 1.

If there are additional modules, repeats the same round with each additional module. Consider an example module referred to as Cathy, Alice 502 fits model 502B based on residual 1 to generate fitted model 502B′, sends Cathy residual 1, and receives a response with a different residual 2. Because Cathy has a different feature set and/or a different model, Cathy's model produces a different prediction for the task label (e.g., LOS prediction).

After the above procedure stops at an appropriate stopping time k=K, Alice 502's training stage 2 is suspended, and the prediction stage commences. In the prediction stage, upon arrival of a new feature vector x to both Alice 502 and Bob 504, Alice 502 uses queries Bob 504's prediction results, which may be xw_{b,K} if model 500B is employed or a combination of fitted model 504B′ and fitted model 504B″ otherwise. Because both fitted model 504B′ and 504B″ are based on model 504B, both fitted models produce prediction results indicating a first task label and a second task label for vector x, respectively. Alice 502 combines Bob 504's prediction results with xw_{a,K} if model 500B is employed or a combination of fitted model 502B′ and fitted model 502B″ otherwise. Because both fitted model 502B′ and 502B″ are based on model 502B, both fitted models produce prediction results indicating a third task label and a fourth task label for vector x. Combining the prediction results from Alice 502 in some mathematical manner results in Alice 502's final prediction.

In the context of LOS prediction example, model 502B′ and model 502B″ produce first and second fitted predictions of the new patient's length of stay but if model 500B is employed, Alice 502 produces prediction results as a vector product of new feature vector x and weight vector w_{a,K} where a weighted value is a prediction of the new patient's length of stay. Similarly, Bob 504's model 504B′ and model 504B″ produce third and fourth fitted predictions of the new patient's length of stay but if model 500B is employed, Bob 504 produces prediction results as a vector product of new feature vector x and weight vector w_{b,K} where a weighted value is a prediction of the new patient's length of stay. While there are several ways to combine predictions from other modules, one method is to use unweighted summation to combine predictions from Alice 502 and Bob 504.

There are a number of alternatives and/or extensions for assisted learning protocol 500A. To let Alice 502 and Bob 504 simultaneously assist each other, separately run two instances of assisted learning protocol 500A where Alice 502 learns from Bob 504 in one instance and Bob 504 learns from Alice 502 in another instance. If Alice 502 is not cooperative after Bob 504 assists Alice 502 in the training stage, Bob 504 no longer assists Alice 502 in the prediction stage. As another solution, assisted learning protocol 500A may be compatible with mechanisms to bind entities together, so that each one must assist others while it is being assisted.

In one implementation, Bob 504 injects a function of y_{B} when Alice 502 initializes assisted learning protocol 500A with an initial label set and/or Alice 502 injects a function of y_{A} when Bob 504 initializes assisted learning protocol 500A with an initial label set. If Alice 502 initializes a set of labels for model 502B, Bob 504 adds values of function y_{B} to the initial labels and trains fitted model 504B′. Alice 502 and Bob 504 may have to jointly decode during the prediction stage, otherwise the prediction may not be technically feasible.

Instead of or in addition to residual passing as described herein, Alice 502 and Bob 504 may exchange confidence scores, such as a confidence score indicating model confidence or object confidence. An example confidence score for model 502B or model 504B may indicate how much of datasets 502A or 504A have been modeled.

As another extension, Alice 502 and Bob 504 may enhancing model privacy by the following information distortion techniques. Alice 502 may include a first (effective) system for public use and a second (authentic) system for private use where the second system is embedded in the first system. The functionality of the effective system is described as follows. For data that are intended to be the input to the authentic system, Alice will first distort them by adding random noises. The perturbed input is then passed into the authentic system. Alice will then distort the output to construct the final output of the effective system. Thus, the effective system is designed to safeguard the internal authentic system from being reverse-engineered by adversarial queries. An example information distortion technique is to design the random noise by minimizing the distance between the effective system and authentic system plus a rescaled mutual information between the perturbed input/output and original input/output.

The techniques described in this disclosure may be implemented, at least in part, in hardware, software, firmware or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

What is claimed is:

1. A method comprising:
creating, by processing circuitry of a computing device, a learner unit by fitting, into a first fitted label set, an initial label set using at least one first learning technique, a first machine learning model, and a first feature set;
sending, by the processing circuitry of the computing device, to at least one module in a machine learning architecture, first statistical information defined by at least one first residual from fitting the first fitted label set, wherein the at least one module is operative to fit, into at least one second fitted label set, the first statistical information using at least one second learning technique, at least one second machine learning model, and at least one second feature set, wherein each of the at least one module executes on at least one remote computing device;
receiving, by the processing circuitry of the computing device, and from the at least one module, second statistical information that is defined by at least one second residual from fitting the second fitted label set; and
updating, by the processing circuitry of the computing device, the learner unit by fitting, into a third fitted label set, the second statistical information using the at least one first learning technique and the first machine learning model.

2. The method of claim 1, further comprising:
generating, from a new feature set and the learner unit, a first set of predicted labels.

3. The method of claim 2, further comprising:
querying the at least one module for a second set of predicted labels for the new feature set.

4. The method of claim 3, further comprising:
combining the first set of predicted labels and the second set of predicted labels into a final set of predicted labels.

5. The method of claim 1, further comprising:
repeating the sending and the receiving until an out-sample error satisfies a criterion, wherein the out-sample error is computed by cross-validation.

6. The method of claim 1, wherein the learner unit and the at least one module implement aligned or partially aligned feature datasets.

7. The method of claim 1, further comprising:
selecting the at least one module to run in the machine learning architecture based on at least one of communication bandwidth, cost constraints, or computational overhead.

8. The method of claim 1,
wherein creating, by the processing circuitry of the computing device, the learner unit further comprises training the first machine learning model using the at least one first learning technique with the initial label set and the first feature set,
wherein the trained machine learning model is configured to generate the first fitted label set for the first feature set,
wherein sending, by the processing circuitry of the computing device, to the at least one module in the machine learning architecture, the first statistical information further comprises determining a first particular residual of the at least one first residual based on a first fitted label of the first fitted label set and at least one of a first initial label of the initial label or first observed data in the first feature set,
wherein the at least one module trains the least one second machine learning model using the at least one second learning technique with the at least one second feature set, wherein a second particular residual of the least one second residual is determined based on a second fitted label of the second fitted label set and at least one of the first particular residual, the first initial label of the initial label set, or first observed data in the second feature set, and
wherein updating, by the processing circuitry of the computing device, the learner unit by fitting, into the third fitted label set, the second statistical information further comprises further training the trained machine learning model with the at least one second residual and the first feature set; and
further comprising sending, by the processing circuitry of the computing device, to the at least one module in the machine learning architecture, third statistical information further defined by at least one third residual from fitting the third fitted label set.

9. The method of claim 8, wherein sending, by the processing circuitry of the computing device, the third statistical information further comprises determining a third particular residual based on a first third fitted label and at least one of the second particular residual of the at least one second residual, the first initial label of the initial label set, or the first observed data in the first feature set.

10. A computing device comprising:
processing circuitry coupled to memory and configured to:
create a learner unit by fitting, into a first fitted label set, an initial label set using at least one first learning technique, a first machine learning model, and a first feature set;
send to at least one module in a machine learning architecture, first statistical information defined by at least one first residual from fitting the first fitted label set, wherein the at least one module is operative to fit, into a second fitted label set, the first statistical information using at least one second learning technique, at least one second machine learning model, and at least one second feature set, wherein each of the at least one module executes on at least one remote computing device;
receive, from the at least one module, second statistical information that is defined by at least one second residual from fitting the second fitted label set; and
update the learner unit by fitting, into a third fitted label set, the second statistical information using the at least one first learning technique and the first machine learning model.

11. The computing device of claim 10, wherein the processing circuitry is further configured to:
send to the at least one module in the machine learning architecture, third statistical information defined by at least one third residual from fitting the third fitted label set using the at least one first learning technique and the first machine learning model, wherein the at least module is operative to fit, into a fourth fitted label set, the third statistical information using the at least one second learning technique, the at least one second machine learning model, and the at least one second feature set.

12. The computing device of claim 11, wherein the processing circuitry is further configured to:
generate, from a new feature set and the learner unit, a first set of predicted labels; and
query the at least one module for a second set of predicted labels for the new feature set, wherein the second set of prediction labels comprises a first predicted label determined by a trained second machine learning model corresponding to the second fitted label set and a second predicted label determined by of a trained second machine learning model corresponding to a fourth fitted label set.

13. The computing device of claim 12, wherein the processing circuitry is further configured to:
combine the first set of predicted labels and the second set of predicted labels into a final set of predicted labels.

14. The computing device of claim 10, wherein the processing circuitry is further configured to:
repeat the sending and the receiving until an out-sample error no longer decreases.

15. The computing device of claim 10, wherein the learner unit and the at least one module implement aligned or partially aligned feature datasets.

16. The computing device of claim 10, wherein the processing circuitry is further configured to:
limit the at least one module to a particular number based on to at least one of communication bandwidth, cost constraints, or computational overhead.

17. The computing device of claim 10, wherein the learner unit and the at least one module implement centralized feature datasets or decentralized feature datasets.

18. The computing device of claim 10, wherein to create the learner unit, the processing circuitry is further configured to:
train the first machine learning model using the at least one first learning technique with the initial label set and the first feature set, wherein the trained machine learning model is configured to generate the first fitted label set for the first feature set;
wherein to send, to the at least one module in the machine learning architecture, the first statistical information, the processing circuitry is further configured to:
determine a first particular residual of the at least one first residual based on a first fitted label of the first fitted label set and an observed data set in the first feature set, wherein the at least one module trains the least one second machine learning model using the at least one second learning technique with the at least one second feature set, wherein a second particular residual of the least one second residual is determined from a second fitted label of the second fitted label set and the observed data set; and
wherein to update the learner unit, the processing circuitry is further configured to:
further train the trained machine learning model with the at least one second residual and the first feature set.

19. A non-transitory, computer-readable medium comprising executable instructions, which when executed by processing circuitry, cause a computing device to perform operations comprising:
creating a learner unit by fitting, into a first fitted label set, an initial label set using at least one first learning technique, a first machine learning model, and a first feature set;
sending, to at least one module in a machine learning architecture, first statistical information defined by at least one first residual from fitting the first fitted label set, wherein the at least one module is operative to fit, into a second fitted label set, the first statistical information using at least one second learning technique, at least one second machine learning model, and at least one second feature set, wherein each of the at least one module executes on at least one remote computing device;
receiving, from the at least one module, second statistical information that is defined by at least one second residual from fitting the second fitted label set; and
updating the learner unit by fitting, into a third fitted label set, the second statistical information using the at least one first learning technique and the first machine learning model.

20. The non-transitory, computer-readable medium of claim 19, wherein the operations further comprise:
generating, from a new feature set and the learner unit, a first set of predicted labels;
querying the at least one module for a second set of predicted labels for the new feature set; and
combining the first set of predicted labels and the second set of predicted labels.
